(19) United States
(12) Patent Application Publication
(10) Pub. No.: US 2010/0076199 A1
(43) Pub. Date: Mar. 25, 2010

(54) PROCESS FOR THE PREPARATION OF SUBSTITUTED PYRIDONE CARBOXYLIC ACIDS

(75) Inventors: VIJAYA RAJ KUNIYIL KULANGARA, Bangalore (IN); TILAK T. RAJ, BANGALORE (IN)

Correspondence Address: GE HEALTHCARE, INC., IP DEPARTMENT 101 CARNEGIE CENTER, PRINCETON, NJ, 08540-6231, US

(73) Assignee: GE HEALTHCARE AS, OSLO (NO)

(21) Appl. No.: 12/561,481

(22) Filed: Sep. 17, 2009

(30) Foreign Application Priority Data
Sep. 24, 2008 (EP) 08164963.4

(51) Int. Cl. C07D 213/69

(52) U.S. Cl. 546/296

(57) ABSTRACT

The present invention relates to methods for the preparation of pyridone carboxylic acids for use as intermediates in the preparation of various synthetic organic compounds. The pyridone carboxylic acids can for example be used as intermediates in the production of potent antitumor agents, antifungal agents, antiviral agents, psychotherapeutic agents or contrast imaging agents for MRI.

### FIELD OF THE INVENTION

The present invention relates to methods for the preparation of pyridone carboxylic acids for use as intermediates in the preparation of various synthetic organic compounds.

### BACKGROUND OF THE INVENTION

Pyridone carboxylic acids are used in various synthesises in organic chemistry, for example as intermediates in the production of potent antitumor agents (Schultz, A. Chem. Rev. 1973, 73, 385; Kelly, T et al., J. Am. Chem. Soc. 1988, 110, 6471; Curran, D et al., J. Am. Chem. Soc. 1992, 114, 5863), antifungal agents (Cox, R et. Al., J. Chem. Soc., Perkin Trans. 1 1991, 2537), antiviral agents (Williams, D et al., Tetrahedron Lett. 1997, 38, 327), psychotherapeutic agents (Kozikowski, A et al., J. Am. Chem. Soc. 1996, 118, 11357) and contrast imaging agents for MRI.

Pyridone carboxylic acids can be prepared by various synthetic pathways known from the literature. The conversion of pyranones to pyridones is well known and is generally done by treating pyranones with the amine of interest.

Pace P et al., Bioorg. Med. Chem. Lett. 2004, 14, 3257 provides a synthesis of 3-Benzyloxypyran-4(1H)-one-2-carboxylic acid starting from maltol (2-methyl-3hydroxypyran-4(1H)-one). The 3-hydroxyl group of maltol is benzyl protected, followed by oxidation of the 2-methyl group in two steps to give 3-benzyloxypyran-4(1H)-one-2-carboxylic acid. The oxidation is carried out first with SeO₂ in bromobenzene and then with sodium chlorite-sulphamic acid system and overall yield reported is 70%.

Liu Z D et al., Bioorg. Med. Chem. 2001, 9, 563 provides a synthesis of 6-methyl-3-benzyloxy-pyridin-4(1H)-one-2-carboxamide starting from 2-methyl-5-hydroxy-pyridin-4(1H)-one. A hydroxyl methanol group was introduced at 2-position that was then oxidized to aldehyde and then to carboxylic acid. Pyridone carboxylic acids were converted into a corresponding amide and then treated with methylamine to get 1-methylpyridine derivatives.

Common for all known methods of preparing pyridone carboxylic acids described in the literature are that they suffer from poor yields, low purity and are very time consuming raising the overall expenses and delivery time.

Therefore, there is a need to provide an improved method of preparing pyridone carboxylic acids that overcomes one or more of the above mentioned problems.

It has now surprisingly been found that the above mentioned problems can be solved by replacing reagents used in prior art with alternative reagents in all three steps of the synthesis.

### DETAILED DESCRIPTION OF THE INVENTION

In a first aspect the present invention provides a method for the preparation of pyridone carboxylic acids of formula (I)

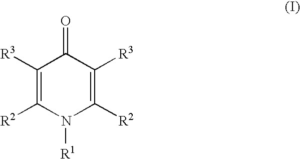

wherein
one R² denotes H and one R² denotes COOH;
one R³ denotes H and one R³ denotes —OR^{P} wherein R^{P} denotes a protecting group and;
R¹ denotes alkyl, alkoxyalkyl, benzyloxyalkyl, di(benzyloxy)alkyl, tri(benzyloxy)alkyl or tetra(benzyloxy)alkyl
comprising the steps of:
a) reacting a pyranone of formula (II) with an amine of formula R¹NH₂ wherein R¹ is as defined above or R^{1′}NH₂ wherein R^{1′} denotes hydroxylated alkyl or hydroxylated alkoxyalkyl in alcoholic medium together with a catalyst comprising sodium hydroxide

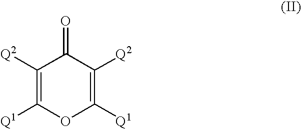

wherein
one Q¹ denotes H and one Q¹ denotes —CH₃;
one Q² denotes H and one Q² denotes —OR^{P} wherein R^{P} is defined as above, and when applicable protecting said hydroxyl groups in R^{1′} with benzyl groups, to form a compound of formula (III)

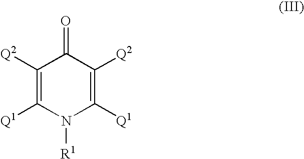

wherein Q¹, Q² and R¹ are defined as above and;
b) reacting the pyridone of formula (III) in a mixture of acetic acid and acetic anhydride with SeO₂ to form a compound of formula (IV)

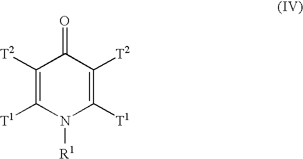

wherein
one T¹ denotes H and one T¹ denotes —CHO;
one T² denotes H and one T² denotes —OR^{P} wherein R^{P} is defined as above and;
c) reacting the pyridone aldehyde of formula (IV) with alkalimetal peroxomonosulfate to form the compound of formula (I).

In the compounds of formulae (I), (II), (III) and (IV) above the protecting group R^{P} can preferably be chosen from C₁-C₄ alkyl, benzyl, tosyl, mesyl or trimethylsilyl, with benzyl being most preferred. The alkyl moieties in the R¹ substituents are preferably a C₁-C₆ alkyl.

The method of the present invention involves improvements over prior art in all three steps of the method described resulting in a less time consuming process and a product with higher yield and purity.

In step a) the pyranone of formula (II) is treated with an amine in alcoholic medium together with sodium hydroxide as catalyst.

The reaction with an amine in alcoholic medium opens the ring structure of pyranone and examination of the reaction mechanism reveals that the reaction usually proceeds through an additional elimination mechanism (Tetrahedron Letters, Vol. 38, No. 40, pp. 7003-7006, 1997). It has now surprisingly been found that sodium hydroxide can catalyse this reaction by stabilising the intermediate anion. Treating the pyranone with a mixture of catalytic amount of sodium hydroxide, the amine and aqueous alcohol increases the yield and purity. In addition the reaction time can be reduced from about 18 to 20 hours reported in prior art to about 12 to 16 hours, and the yield can be increased to about 76 to 94% from about 42% experienced with methods according to prior art.

The alcohol used can be any type of aliphatic alcohol, preferably a C₁-C₆ alcohol and most preferably methanol or ethanol.

The pyranones used in step a) are commercially available. Preferably the pyranones are chosen from the compounds of formula (IIa) and (IIb), wherein R^{P} is defined as above.

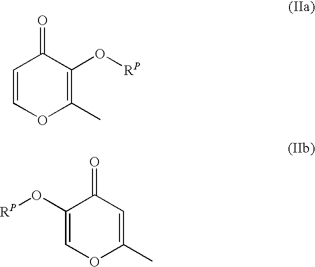

The amines used in step a) are commercially available and of formula R¹NH₂ or R^{1′}NH₂ as defined above. Said amines are preferably selected from

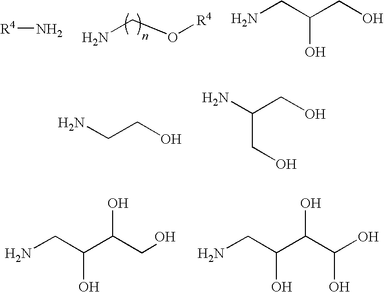

wherein R⁴ is a C₁-C₆ straight or branched alkyl and;
n denotes a positive integer from 1 to 6.

Most preferably the amine is 3-amino-1,2-dihydroxypropane or ethanolamine.

In the preferred method of carrying out step a) of the invention, pyranone and amine can be added in a weight ratio of about 1:0.75 to 1:1.5, preferably about 1:1. NaOH, preferably about 2N NaOH, can be added at a ratio of 6 to 9 ml per 0.1 mole of pyranone, preferably about 8.25 ml. Alcohol can preferably be added in an amount of about 5 to about 10 ml per gram pyranone, and water preferably in an amount of about 2.5 to about 5 ml per gram pyranone.

The reaction is conducted at reflux for about 14 to 18 hours, preferably about 16 hours.

After the reaction in step a) is completed or stopped, the product is optionally purified and isolated by conventional methods, e.g. extraction, washing, distillation, crystallisation etc.

It has now surprisingly been found that by using a mixture of acetic acid and acetic anhydride instead of bromobenzene allows for the production of formula (IV). The reaction time is reduced, the reaction temperature can be brought down and the yield and purity are increased.

Therefore, in step b) the compound of formula (III) from step a) in a mixture of acetic acid and acetic anhydride and is reacted with SeO₂.

Oxidation of allylic methyl groups to aldehyde using SeO₂ is known in prior art, but applying these methods to pyridones results in poor yield and purity. The reaction rate is low in spite of high reaction temperature, which results in low purity. The higher boiling point of bromobenzene used in prior art, and its water immiscible nature, create problems during work up.

In the preferred method of carrying out step b) of the invention the mixture of acetic acid and acetic anhydride can preferably comprise acid and anhydride in the weight ratio of about 1:1 to 1:1.5, most preferably about 1:1. SeO₂ can preferably be added in an amount of about 1.2 to 1.8 mole per mole of pyridone.

Preferably the reaction is conducted at a temperature from about 50 to about 110° C. with stirring for about 2 to 10 hours, preferably at about 80° C. with stirring for about 4 hours.

After the reaction in step b) the product can optionally be purified and isolated by conventional methods, e.g. extraction, washing etc.

It has now surprisingly been found that favourable reaction conditions and work up are achieved when using an oxidation agent comprising alkalimetal peroxomonosulfate. The yield and purity are also increased considerably.

Hence, in step c) the pyridone aldehyde of formula (IV) from step b) is reacted with alkalimetal peroxomonosulfate.

Oxidation of aldehydes to acids can be performed in many ways according to prior art. Generally potassium permanganate, potassium dichromate and sodium hypochlorite are common reagents for this conversion but involve time-consuming work up procedures.

Preferably, the alkalimetal peroxomonosulfate is potassium peroxomonosulfate. Potassium peroxomonosulfate is commercially available under the trademark Oxone®.

In the preferred method of carrying out step c) of the invention pyridone aldehyde in DMF (dimethylformamide) is reacted with potassium peroxomonosulfate. Potassium peroxomonosulfate is preferably added in an amount of about 1.0 to about 1.2 mole per mole of the pyridone aldehyde.

Preferably the reaction is conducted at a temperature of about 0 to about 50° C. with stirring for about 4 to about 16 hours, preferably at room temperature with stirring for about 12 hours.

After the reaction in step c) the product can optionally be purified and isolated by conventional methods, e.g. extraction, chromatography etc.

The compounds prepared by the method of the invention can for example be used as starting material in the production of tripodal chelates of general formula (V), designed for the purpose of making chelating agents that can form complexes with paramagnetic metal ions to obtain paramagnetic metal chelates

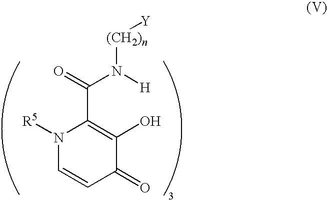

wherein
R⁵ is C₁₋₆ alkyl, optionally substituted with 1 to 4 OH— or O—C₁₋₆ alkyl-groups; or polyethylene glycol of up to 4 monomer units.
n denotes a positive integer from 1 to 4; and
Y is a trivalent group.

By trivalent group is meant a group with three functional groups that makes attachment to three other groups possible.

Preferred trivalent groups with amines as functional groups are

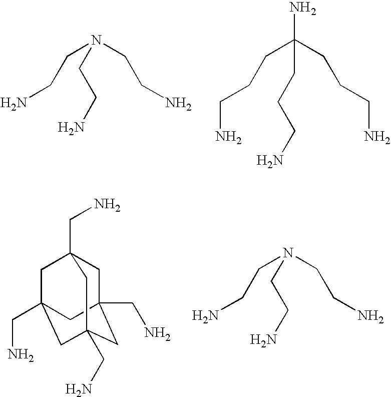

In a second aspect the present invention provides a method for preparing a pyridone of formula (III) comprising reacting a pyranone of formula (II) with an amine in alcoholic medium employing a catalyst comprising sodium hydroxide.

The resulting pyridone is a useful intermediate in the production of pyridone acids described above.

In a third aspect the present invention provides a method for preparing a pyridone aldehyde of formula (IV) comprising reacting a pyridone of formula (III) with a mixture of acetic acid and acetic anhydride and SeO₂.

The resulting pyridone aldehyde is a useful intermediate in the production of pyridone acids described above.

In a fourth aspect the present invention provides a method for preparing a pyridone carboxylic acid of formula (I) comprising reacting the corresponding pyridone aldehyde of formula (IV) with alkalimetal peroxomonosulfate.

The present invention will now be further illustrated by way of the following non-limiting examples.

### EXAMPLES

The following abbreviations are used:

THF denotes tetrahydrofuran
DMF denotes dimethylformamide
TLC denotes thin layer chromatography
HPLC denotes high performance liquid chromatography

### Example 1

### Synthesis of 3-Benzyloxy-1-(2,3-dibenzyloxypropyl)-pyridin-4(1H)-one-2-carboxylic acid

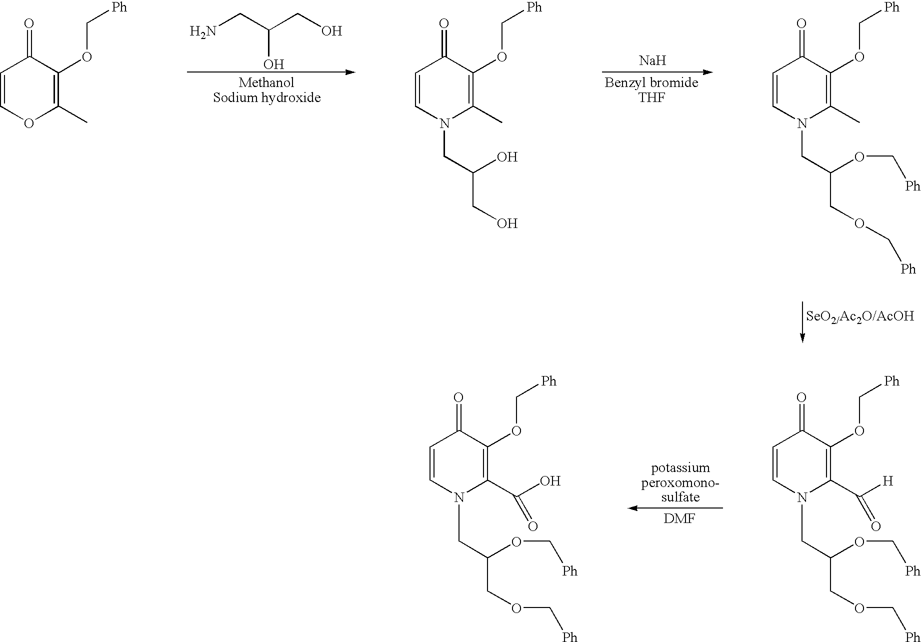

### (a) Preparation of 3-benzyloxy-2-methyl-1-(propane-2,3-diol)-pyridin-4(1H)-one

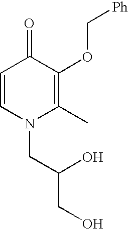

3-Benzyloxy-2-methyl-pyran-4-one (30 g), methanol (300 ml) and water (75 ml) was treated with 3-amino-1,2-dihydroxypropane (31.6 g) and 2N sodium hydroxide (11.4 ml) at reflux for 16 h. The reaction was monitored by TLC on silica in 25% methanol in ethyl acetate. The reaction was then concentrated in vacuum to remove the methanol and the product was extracted into dichloromethane (300 ml). The dichloromethane layer was separated, washed with brine solution (50 ml) followed by water (50 ml), dried over anhydrous sodium sulphate and the solvent removed in vacuum. The remaining water was removed by azeotropic distillation with toluene (50 ml). The product was crystallized from ethyl acetate (20 ml) and acetone (80 ml) as a light yellow solid (30.5 g, 76%); purity by HPLC 99.46%.

¹H-NMR (CDCl₃): δ2.2 (s, 3H, CH₃), 3.5 (t, 2H, CH₂), 3.9 (t, 2H, CH₂), 4.2 (m, 1H, CH), 5.1 (s, 2H, CH₂), 6.4 (d, 1H, ArH), 7.3-7.5 (m, 5H, ArH), 7.7 (d, 1H, ArH);

MS: 290.5 (M+1)

### (b1) Preparation of 3-Benzyloxy-2-methyl-1-(2,3-(dibenzyloxy)propyl)-pyridin-4(1H)-one

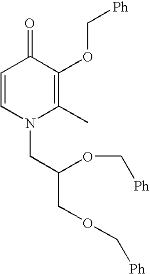

3-Benzyloxy-2-methyl-1-(2,3-dihydroxypropyl)-pyridin-4(1H)-one (20.0 g) in THF (200 ml) was stirred with sodium hydride powder (7.0 g) and treated drop-wise with benzyl chloride (19.26 g), and the reaction mixture was refluxed for 18 h. The reaction was monitored by TLC on silica run in 25% methanol in ethyl acetate. On completion the reaction was carefully quenched with water (2000 ml) and the product recovered by extraction into dichloromethane (400 ml). The dichloromethane layer was separated, dried over anhydrous sodium sulphate, and concentrated in vacuum to a light brown solid. This was purified by column chromatography on silica eluting in a gradient of ethyl acetate in hexane to give the title compound (24.3 g, 75%); purity by HPLC 97.06%

¹H-NMR (CDCl₃): δ2.1 (s, 3H, CH₃), 3.5 (t, 2H, CH₂), 3.9 (t, 2H, CH₂), 4.2 (m, 1H, CH), 4.2 (s, 2H, CH₂), 4.5 (s, 2H, CH₂), 4.8 (s, 2H, CH₂), 6.4 (d, 1H, ArH), 7.3-7.5 (m, 15H, ArH), 7.6 (d, 1H, ArH); MS: 470.6 (M+1)

### (b2) Preparation of 3-Benzyloxy-1-(2,3-dibenzyloxypropyl)-pyridin-4(1H)-one-2-carboxaldehyde

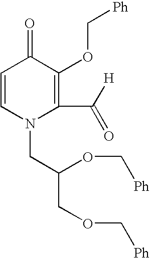

3-Benzyloxy-2-methyl-1-(1,2-dibenzyloxypropyl)-pyridin-4(1H)-one (10 g) in acetic acid (40 ml) and acetic anhydride (40 ml) was treated with selenium dioxide (4.25 g) at 80° C. for 4 h with stirring. The reaction was monitored by TLC on silica in 10% methanol in ethyl acetate. The reaction mixture was diluted with water (1.0 L) and the product recovered by extraction into ethyl acetate (200 ml). The ethyl acetate layer was washed with brine solution (50 ml) followed by water (50 ml) and dried over anhydrous sodium sulphate and concentrated to a brown solid. Chromatography on silica in 20% hexane in ethyl acetate gave the title compound (7.7 g, 75%); purity by HPLC 98.9%

¹H-NMR (CDCl₃): 3.5 (t, 2H, CH₂), 3.9 (t, 2H, CH₂), 4.2 (m, 1H, CH), 4.2 (s, 2H, CH₂), 4.5 (s, 2H, CH₂), 4.8 (s, 2H, CH₂), 6.4 (d, 1H, ArH), 7.3-7.5 (m, 15H, ArH), 7.6 (d, 1H, ArH), 10.2 (s, 1H, CHO); MS: 484.5 (M+1)

### (c) Preparation of 3-Benzyloxy-1-(2,3-dibenzyloxypropyl)-pyridin-4(1H)-one-2-carboxylic acid

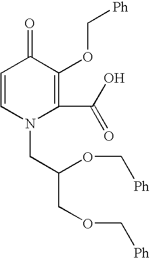

3-Benzyloxy-1-(1,2-dibenzyloxypropyl)-pyridin-4(1H)-one-2-carboxaldehyde (9.0 g) in DMF (90 ml) was treated with of potassium peroxomonosulfate (12.6 g) at room temperature for 12 h with stirring. The reaction was monitored by TLC on silica in 25% methanol in ethyl acetate. After completion of the reaction, reaction mixture was diluted with water (90 ml), cooled to 10° C. and acidified to pH 2.0-3.0 with concentrated HCl. The solid was filtered and re-dissolved in sodium bicarbonate solution, filtered and acidified again to pH 2.0-3.0 with concentrated. HCl to give the product as white amorphous solid (4.2 g, 45.6%); Purity by HPLC 98.36%

¹H-NMR (CDCl₃): 3.5 (t, 2H, CH₂), 3.9 (t, 2H, CH₂), 4.2 (m, 1H, CH), 4.2 (s, 2H, CH₂), 4.5 (s, 2H, CH₂), 5.1 (s, 2H, CH₂), 6.3 (d, 1H, ArH), 7.2-7.5 (m, 15H, ArH), 7.6 (d, 1H, ArH); MS: 500.5 (M+1)

### Example 2

### Synthesis of 3-Benzyloxy-1-(2-benzyloxyethyl)-pyridin-4(1H)-one-2-carboxylic acid

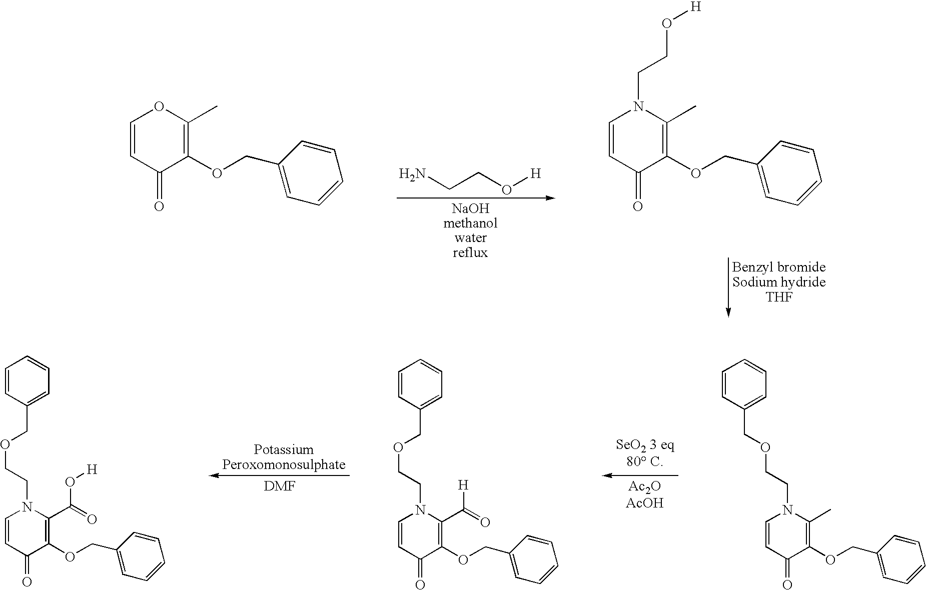

### (a) Preparation of 2-methyl-1-(ethane-2-ol)-3-benxyloxy-pyran-4-(1H)-one

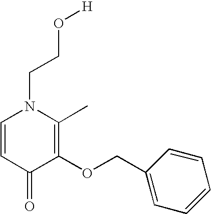

3-Benzyloxy-2-methyl-pyran-4-one (21.6 g) in aqueous methanol (200 ml) was treated with ethanolamine (12.2 g) and 2N aqueous sodium hydroxide (8.2 ml) at reflux temperature for 16 h. The reaction was monitored by TLC on silica in 25% methanol in ethyl acetate. The reaction was then concentrated in vacuum to remove the methanol and the product was recovered by extraction into dichloromethane (200 ml). The dichloromethane solution was washed with brine (50 ml) followed by water (50 ml), dried over anhydrous sodium sulphate and the solvent removed in vacuum. The residue was crystallized from mixture of hexane (50 ml) and acetone (50 ml) to give the title compound as an off-white solid (22.2 g, 86%). Purity by HPLC 98%

¹H-NMR (CDCl₃): δ2.1 (s, 3H, CH₃), 3.8 (s, 4H, CH₂CH₂), 4.9 (s, 2H, CH₂), 6.2 (d, 1H, ArH), 6.6 (bs, 1H, OH), 7.2-7.4 (m, 6H, ArH); ¹³C-NMR: δ13.0, 58.0, 60, 72, 74, 118, 128.0, 128.7, 129.4, 138, 139, 142, 146, 178,

MS: 260.5 (M+1)

### (b1) Preparation of 2-methyl-1-(2-benzyloxyethyl)-3-benxyloxy-pyran-4-(1H)-one

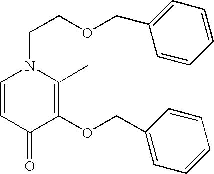

3-Benzyloxy-2-methyl-1-(2-hydroxyethyl)-pyridin-4(1H)-one (20.0 g) in THF (200 ml) was treated with sodium hydride (4.0 g) followed by the drop-wise addition of benzyl bromide (4.5 g). The reaction mixture was refluxed for 6 h. The reaction was monitored by TLC on silica in 25% methanol in ethyl acetate and was quenched by the careful addition of water (200 ml). The product was then extracted with dichloromethane. After separation the organic layer was dried over anhydrous sodium sulphate and concentrated to a light brown residue (26.4 g, 98%). This residue was directly taken for the next stage without any further purification. Purity by HPLC 99.98%

¹H-NMR (CDCl₃): δ2.1 (s, 3H, CH₃), 3.5 (t, 2H, CH₂), 3.8 (t, 2H, CH₂), 4.5 (s, 2H, CH₂), 5.1 (s, 2H, CH₂), 6.4 (d, 1H, ArH), 7.1-7.4 (m, 11H, ArH); MS: 350.38 (M+1)

### (b2) Preparation of 2-formyl-1-(2-benxyloxyethyl)-3-benxyloxy-pyran-4-(1H)-one

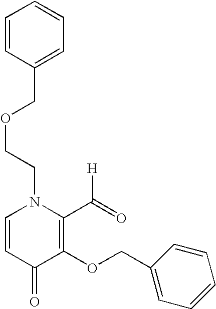

3-Benzyloxy-2-methyl-1-(2-benzyloxyethyl)-pyridin-4(1H)-one (20 g) in acetic aid (100 ml) and acetic anhydride (100 ml) was treated with selenium dioxide (11.5 g) at 80° C. for 4 h under stirring. The reaction was monitored by TLC on silica in 10% methanol in ethyl acetate. The reaction mixture was diluted with water (1.0 L) and the product was extracted with ethyl acetate (200 ml). The Ethyl acetate layer was washed with brine (50 ml) followed by water (50 ml) and dried over anhydrous sodium sulphate. The ethyl acetate layer was concentrated to a brown residue. The residue was purified by column chromatography on silica eluting with 5% methanol in ethyl acetate to give the title compound (13.0 g, 62.5%). Purity by HPLC 90%

¹H-NMR (CDCl₃): 3.5 (t, 2H, CH₂), 3.8 (t, 2H, CH₂), 4.4 (s, 2H, CH₂), 5.0 (s, 2H, CH₂), 6.4 (d, 1H, ArH), 7.1-7.4 (m, 11H, ArH), 10.1 (s, 1H, CHO);

MS: 364.43 (M+1)

### (c) Preparation of 1-(2-benxyloxyethyl)-3-benxyloxy-pyran-4-(1H)-one-2-carboxylic acid

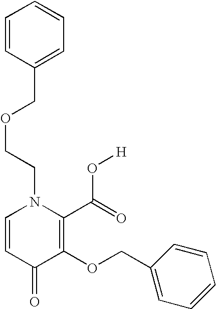

3-Benzyloxy-1-(2-benzyloxyethyl)-pyridin-4(1H)-one-2-carboxaldehyde (12.0 g) in DMF (120 ml) was treated with potassium peroxomonosulfate (21.0 g) at room temperature for 12 h under stiffing. The reaction was monitored by TLC on silica in 25% methanol in ethyl acetate. After completion of the reaction mixture was diluted with water (120 ml), cooled to 10° C. and acidified to pH 2.0-3.0 with con. HCl. The solid was filtered and re-dissolved in sodium bicarbonate solution, filtered and acidified again to pH 2.0-3.0 with con. HCl to give the title product as a white amorphous solid (6.4 g, 51.2%) Purity by HPLC 99%

¹H-NMR (CDCl₃): 3.7 (t, 2H, CH₂), 4.1 (t, 2H, CH₂), 4.45 (s, 2H, CH₂), 5.15 (s, 2H, CH₂), 6.4 (d, 1H, ArH), 7.2-7.4 (m, 10H, ArH), 7.7 (d, 1H, ArH); MS: 380.5 (M+1)

### Example 3

### Synthesis of 3-Benzyloxy-1-(propyl)-pyridin-4(1H)-one-2-carboxylic acid

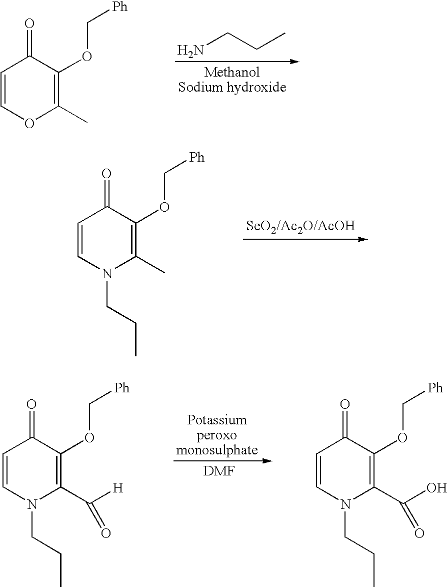

### (a) Preparation of 3-Benzyloxy-2-methyl-1-(propyl)-pyridin-4(1H)-one

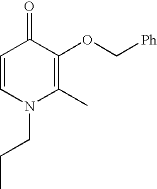

To a solution of 25 g (0.1156 mole) of 3-benzyloxy-2-methyl-pyran-4-one in aqueous methanol (75 ml+75 ml) was added 27.3 g (0.4736 mole) of N-propylamine followed by 9.5 ml 2N NaOH solution. The reaction mixture was refluxed (set temp 80° C.) for 12-14 h. Progress of the reaction was monitored by TLC run in 25% methanol in ethyl acetate. After completion of the reaction, methanol was distilled off completely and the product was extracted to dichloromethane (250 ml) and washed with brine solution (50 ml) followed by water (50 ml). After separation the organic layer was dried over anhydrous sodium sulphate and concentrated to dark yellow oil. This was directly taken for the next stage without any further purification (28.0 g, 93.7%); Purity by HPLC 99.8%

¹H-NMR (CDCl₃): δ0.9 (q, 3H, CH₃), 1.6 (p, H, CH₂), 2.1 (s, 3H, CH₃), 3.65 (t, 2H, CH₂), 5.2 (s, 2H, CH₂), 6.4 (d, 1H, ArH), 7.1 (d, 1H, ArH), 7.2-7.4 (m, 5H, ArH); ¹³C-NMR (CDCl₃): δ10.1, 13.0, 25.0, 55.1, 73.0, 117, 128, 129, 130, 138, 139, 142, 147, 174; MS: 258 (M+1)

### (b) Preparation of 3-Benzyloxy-1-(propyl)-pyridin-4(1H)-one-2-carboxaldehyde

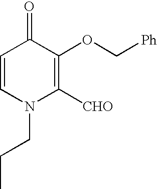

To a solution of 10 g (0.0388 mole) of 3-Benzyloxy-2-methyl-1-(propyl)-pyridin-4(1H)-one in acetic aid (50 ml) and acetic anhydride (50 ml) was added 8.0 g (0.0720 mole) of selenium dioxide. The reaction mixture was kept at 100° C. for 4 h under stiffing. Progress of the reaction was monitored by TLC (Mobile phase: 10% methanol in ethyl acetate). After completion of the reaction, reaction mixture was diluted with water (1.0 L) and the product was extracted to ethyl acetate (100 ml), washed with brine solution (50 ml) followed by water (50 ml) and dried over anhydrous sodium sulphate. The ethylacetate layer was concentrated to a residue. The residue is taken directly for the net stage without purification (10.5 g, 100%); Purity by HPLC 71.2%

¹H-NMR (CDCl₃): δ0.9 (q, 3H, CH₃), 1.6 (p, 2H, CH₂), 4.1 (t, 2H, CH₂), 5.5 (s, 2H, CH₂), 6.6 (d, 1H, ArH), 7.3 (d, 1H, ArH), 7.4-7.5 (m, 5H, ArH), 10.1 (s, 1H, CHO); MS: 272 (M+1)

### (c) Preparation of 3-Benzyloxy-1-(propyl)-pyridin-4(1H)-one-2-carboxylic acid

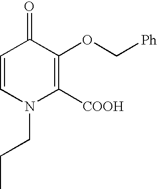

To a solution of 5.0 g (0.0184 mole) of 3-Benzyloxy-1-(propyl)-pyridin-4(1H)-one-2-carboxaldehyde in DMF (50 ml) was added 11.3 g (0.0184 mole) of potassium peroxomonosulfate. The reaction mixture was kept at room temperature for 12 h under stirring. Progress of the reaction was monitored by TLC (mobile phase: 25% methanol in ethyl acetate). After completion of the reaction, reaction mixture was diluted with water (50 ml), cooled to 10° C. and acidified to pH 2.0-3.0 with concentrated HCl. The solid was filtered and re-dissolved in sodium bicarbonate solution, filtered and acidified again to pH 2.0-3.0 with con. HCl to get the product as white amorphous solid (1.6 g, 30%); Purity by HPLC 99% ¹H-NMR (CDCl₃): δ0.9 (q, 3H, CH₃), 1.6 (p, H, CH₂), 4.1 (t, 2H, CH₂), 5.5 (s, 2H, CH₂), 6.6 (d, 1H, ArH), 7.3 (d, 1H, ArH), 7.4-7.5 (m, 5H, ArH), 10.1 (s, 1H, CHO); MS: 272 (M+1)

### Example 4

### Synthesis of 3-Benzyloxy-1-(2-methoxyethyl)-pyridin-4(1H)-one-2-carboxylic acid

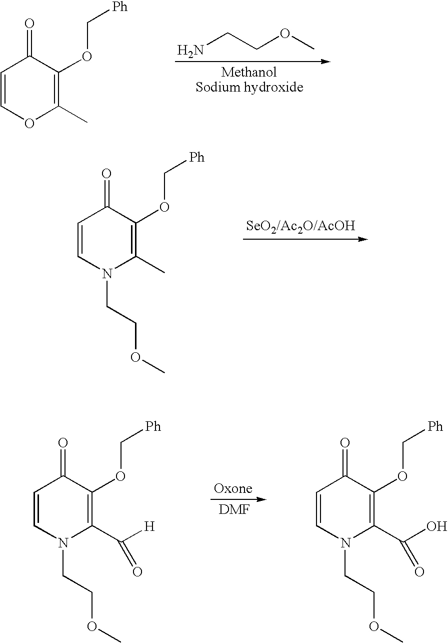

### (a) Synthesis of 3-Benzyloxy-2-methyl-1-(2-methoxyethyl)-pyridin-4(1H)-one

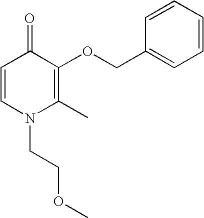

To a solution of 25 g (0.1156 mole) of 3-benzyloxy-2-methyl-pyran-4-one in aqueous methanol (75 ml+75 ml) was added 27.3 g (0.4736 mole) of 2-methylethylamine, 17.4 g (0.2312 mole) followed by 9.5 ml 2N NaOH solution. The reaction mixture was refluxed (set temp 80° C.) for 12-14 h. Progress of the reaction was monitored by TLC (mobile phase: 25% methanol in ethyl acetate). After completion of the reaction, methanol was distilled off completely and the product was extracted to dichloromethane (250 ml) and washed with 50 ml brine solution. After separation, the organic layer was dried over anhydrous sodium sulphate and concentrated to dark yellow oil. This was directly taken for the next stage without any further purification (28.0 g, 93.7%); Purity by HPLC 96.0%.

¹H-NMR (CDCl₃): δ2.1 (s, 3H, CH₃), 3.2 (s, 3H, CH₃), 3.5 (t, 2H, CH₂), 3.8 (t, 2H, CH₂), 5.2 (s, 2H, CH₂), 6.4 (d, 1H, ArH), 7.3 (d, 1H, ArH), 7.4-7.5 (m, 5H, ArH); ¹³C-NMR: δ 3.0, 58.0, 60, 72, 74, 118, 128.0, 128.7, 129.4, 138, 139, 142, 146, 178, MS: 274.12 (M+1)

### (b) Synthesis of 3-Benzyloxy-1-(2-methoxyethyl)-pyridin-4(1H)-one-2-carboxaldehyde

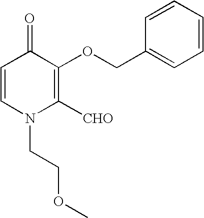

To a solution of 10 g (0.0366 mole) 3-Benzyloxy-2-methyl-1-(2-methoxyethyl)-pyridin-4(1H)-one in acetic aid (50 ml) and acetic anhydride (50 ml) was added 7.3 g (0.0657 mole) of selenium dioxide. The reaction mixture was kept at 100° C. for 4 h under stiffing. Progress of the reaction was monitored by TLC (mobile phase: 10% methanol in ethyl acetate). After completion of the reaction, the reaction mixture was diluted with water (1.0 L) and the product was extracted to ethyl acetate (100 ml), washed with brine solution (50 ml) followed by water (50 ml) and dried over anhydrous sodium sulphate. The ethyl acetate layer was concentrated to a residue. The moisture in the product was removed by azeotropic distillation with toluene and used for the next stage without purification (7.5 g, 71.4%); Purity by HPLC 78%

¹H-NMR (CDCl₃): δ 3.25 (s, 3H, CH₃), 3.5 (t, 2H, CH₂), 4.3 (t, 2H, CH₂), 5.5 (s, 2H, CH₂), 6.5 (d, 1H, ArH), 7.3 (d, 1H, ArH), 7.2-7.5 (m, 5H, ArH), 10.0 (s, 1H, CHO); MS: 288 (M+1)

### (c) Preparation of 3-Benzyloxy-1-(2-methoxyethyl)-pyridin-4(1H)-one-2-carboxylic acid

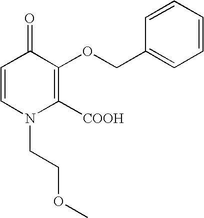

To a solution of 7.0 g (0.0248 mole) of 3-Benzyloxy-1-(2-methoxyethyl)-pyridin-4(1H)-one-2-carboxaldehyde in DMF (50 ml) was added 15.0 g (0.0248 mole) of potassium peroxomonosulfate. The reaction mixture was kept at room temperature for 12 h under stirring. Progress of the reaction was monitored by TLC (Mobile phase: 25% methanol in ethyl acetate). After completion of the reaction, reaction mixture was diluted with water (70 ml), cooled to 10° C. and acidified to pH 2.0-3.0 with concentrated HCl. The solid was filtered and re-dissolved in sodium bicarbonate solution, filtered and acidified again to pH 2.0-3.0 with concentrated HCl to get the product as white amorphous solid (2.8 g, 38%); Purity by HPLC 99%

¹H-NMR (CDCl₃): δ 3.25 (s, 3H, CH₃), 3.5 (t, 2H, CH₂), 4.0 (t, 2H, CH₂), 5.0 (s, 2H, CH₂), 6.4 (d, 1H, ArH), 7.2-7.5 (m, 5H, ArH), 7.6 (d, 1H, ArH), 14.5 (s, 1H, COOH); MS: 304.52 (M+1)

What is claimed is:

1. Process for the preparation of pyridone carboxylic acids of formula (I) 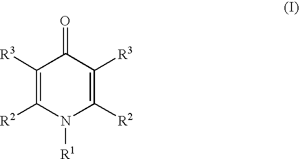
wherein
one R² denotes H and one R² denotes COOH;
one R³ denotes H and one R³ denotes —OR^{P} wherein R^{P} denotes a protecting group and;
R¹ denotes alkyl, alkoxyalkyl, benzyloxyalkyl, di(benzyloxy)alkyl, tri(benzyloxy)alkyl or tetra(benzyloxy)alkyl
comprising the steps of:
a) reacting a pyranone of formula (II) with an amine of formula R¹NH₂ wherein R¹ is defined as above or R^{1′}NH₂ wherein R^{1′} denotes hydroxylated alkyl or hydroxylated alkoxyalkyl in alcoholic medium together with a catalyst comprising sodium hydroxide 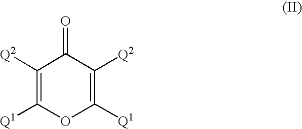
wherein
one Q¹ denotes H and one Q¹ denotes —CH₃;
one Q² denotes H and one Q² denotes —OR^{P} wherein R^{P} is defined as above, and when applicable protecting said hydroxyl groups in R^{1′} with benzyl groups, to form a compound of formula (III) 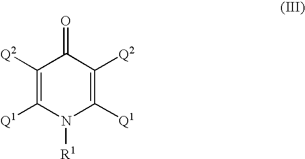
wherein Q¹, Q² and R¹ are defined as above
and;
b) reacting the pyridone of formula (III) in a mixture of acetic acid and acetic anhydride with SeO₂ to form a compound of formula (IV) 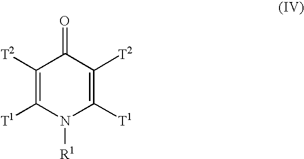
wherein
one T¹ denotes H and one T¹ denotes —CHO;
one T² denotes H and one T² denotes —OR^{P} wherein R^{P} is defined as above
and;
c) reacting the pyridone aldehyde of formula (IV) with alkalimetal peroxomonosulfate to form the compound of formula (I).

2. Process of claim 1 wherein the protecting group R^{P} is selected from C₁-C₄ alkyl, benzyl, tosyl, mesyl or trimethylsilyl, preferably benzyl.

3. Process of claim 1 wherein the alkyl moieties in the R¹ substituents are preferably a C₁-C₆ alkyl.

4. Process of claim 1 wherein the pyranone of formula (II) is selected from one of 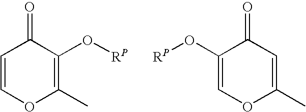
wherein R^{P} is defined as above.

5. Process of claim 1 wherein the amine of formula R¹NH₂ or R^{1′}NH₂ is selected from 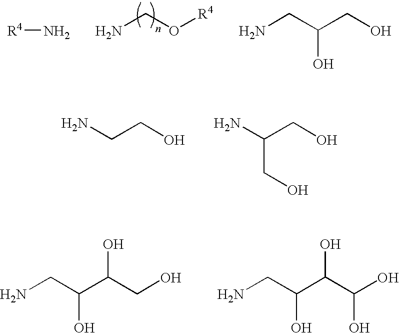
wherein R⁴ is a C₁-C₆ straight or branched alkyl and;
n denotes a positive integer from 1 to 6.

6. Process of claim 5 wherein the amine is selected from 3-amino-1,2-dihydroxypropane and ethanolamine.

7. Process of claim 1 wherein said alcoholic medium in step a) is selected from methanol and ethanol.

8. Process of claim 1 wherein the pyranone of formula (II) and amine of step a) are added in a weight ratio of about 1:0.75 to 1:1.5, preferably about 1:1.

9. Process of claim 1 wherein the alcoholic medium of step a) is added in an amount of about 5 to about 10 ml per gram pyranone.

10. Process of claim 1 wherein sodium hydroxide of about 2N is added at a ratio of 6 to 9 ml per 0.1 mole pyranone.

11. Process of claim 1 wherein the acetic acid and acetic anhydride mixture of step b) is added in a weight ratio of about 1:1 to 1:1.5, preferably 1:1.

12. Process of claim 1 wherein SeO₂ in step b) is added in an amount of about 1.2 to 1.8 mole per mole of pyridone.

13. Process of claim 1 wherein alkali metal peroxomonosulfate of step c) is added in an amount of about 1.0 to 1.2 mole per mole of pyridone aldehyde of formula (IV).

14. Process of claim 1 wherein the alkali metal peroxomonosulfate is potassium peroxomonosulfateoxone.

15. Process for the preparation of pyridones of formula (III) 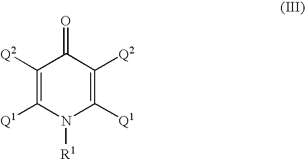
wherein
R¹ denotes alkyl, alkoxyalkyl, benzyloxyalkyl, di(benzyloxy)alkyl, tri(benzyloxy)alkyl or tetra(benzyloxy)alkyl;
one Q¹ denotes H and one Q¹ denotes —CH₃; and
one Q² denotes H and one Q² denotes —OR^{P} wherein R^{P} is a protecting group
by reacting a pyranone of formula (II) 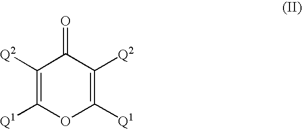
wherein Q¹ and Q² are defined as above
with an amine of formula R¹NH₂ wherein R¹ is defined as above or R^{1′}NH₂ wherein R^{1′} denotes hydroxylated alkyl or hydroxylated alkoxyalkyl in alcoholic medium together with a catalyst comprising sodium hydroxide.

16. Process for the preparation of pyridone aldehydes of formula (IV) 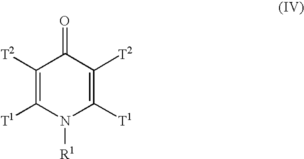
wherein
R¹ denotes alkyl, alkoxyalkyl, benzyloxyalkyl, di(benzyloxy)alkyl, tri(benzyloxy)alkyl or tetra(benzyloxy)alkyl;
one T¹ denotes H and one T¹ denotes —CHO; and
one T² denotes H and one T² denotes —OR^{P} wherein R^{P} is a protecting group.
by reacting the pyridone of formula (III) 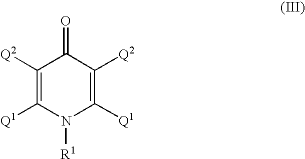
wherein
one Q¹ denotes H and one Q¹ denotes —CH₃;
one Q² denotes H and one Q² denotes —OR^{P} wherein R^{P} is defined as above; and
R¹ is defined as above
in a mixture of acetic acid and acetic anhydride with SeO₂.

17. Process for the preparation of pyridone carboxylic acids of formula (I) 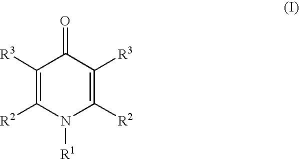
wherein
one R² denotes H and one R² denotes COOH;
one R³ denotes H and one R³ denotes —OR^{P} wherein R^{P} denotes a protecting group; and
R¹ denotes alkyl, alkoxyalkyl, benzyloxyalkyl, di(benzyloxy)alkyl, tri(benzyloxy)alkyl or tetra(benzyloxy)alkyl
by reacting the pyridone aldehyde of formula (IV) 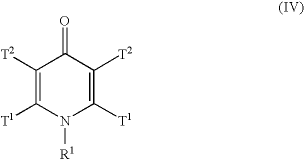
wherein
one T¹ denotes H and one T¹ denotes —CHO;
one T² denotes H and one T² denotes —OR^{P} wherein R^{P} is defined as above; and
R¹ is defined as above
with alkalimetal peroxomonosulfate.
